(19) United States
(12) Patent Application Publication
(10) Pub. No.: US 2008/0241246 A1
(43) Pub. Date: Oct. 2, 2008

(54) Cell-based therapies for treating liver disease

(75) Inventors: Ramasamy Sakthivel, Solon, OH (US); Donald J. Brown, Cleveland, OH (US); Vincent Pompili, Hudson, OH (US); Yukang Zhao, Cleveland Heights, OH (US)

Correspondence Address: ROPES & GRAY LLP, PATENT DOCKETING 39/41, ONE INTERNATIONAL PLACE, BOSTON, MA, 02110-2624, US

(73) Assignee: Arteriocyte Inc., Cleveland, OH (US)

(21) Appl. No.: 11/985,616

(22) Filed: Nov. 15, 2007

Related U.S. Application Data
(60) Provisional application No. 60/859,362, filed on Nov. 15, 2006.

(51) Int. Cl. A61K 9/00; A61K 35/12; A61K 38/20; A61P 1/16

(52) U.S. Cl. 424/486; 424/93.7; 424/484; 424/85.2

(57) ABSTRACT

The invention provides, in part, methods for treating liver disease in a subject in need thereof. The invention further provides methods for improving liver function in a subject in need thereof, such as a subject with an ischemic liver or a cirrhotic liver. In some aspects, the methods of treatment comprise the administration of AC133+ alone or in combination with other cell populations.

### RELATED APPLICATIONS

This application claims the benefit of U.S. Provisional Patent Application 60/859,362, filed Nov. 15, 2006, the entirety of which is incorporated herein by this reference.

### BACKGROUND OF THE INVENTION

Liver disease is categorized both by the cause and the effect it has on the liver. Causes may include infection, injury, exposure to drugs or toxic compounds, an autoimmune process, or a genetic defect (such as hemochromatosis). These causes can lead to hepatitis, cirrhosis, stones that develop and form blockages, fatty liver, and in rare instances liver cancer. Genetic defects can prevent vital liver functions and lead to the deposition and build-up of damaging substances, such as iron or copper.

Ischemic liver causes functional and structural damage to liver cells. Due to the lack of blood (or oxygen-rich blood), ischemia may cause ionic disequilibrium or constant injury of organs or tissues. When ischemia lasts over 10 minutes, reperfusion can cause further injury. Oxidative stress is known as one of the major injuries that occurs mostly when oxygen is reintroduced to ischemia tissue to generate free radicals through conventional Fenton reaction. Liver ischemia/reperfusion injury involving pathogenic shock can occur after liver transplantation and hepatic surgery for trauma or cancer.

Liver cirrhosis, or cirrhosis, is a chronic liver disease in which fibrous tissue and nodules replace normal tissue, interfering with blood flow and normal functions of the organ. Cirrhosis can be caused by, e.g., chronic alcoholism, chronic viral hepatitis (types B, C, and D), cystic fibrosis, severe reactions to prescribed drugs, prolonged exposure to environmental toxins, etc.

Cirrhosis may cause irreversible liver damage. If untreated, liver and kidney failure and gastrointestinal hemorrhage can occur, sometimes leading to death. In the United States, cirrhosis results in about 25,000 deaths annually. Apart from a vegetable protein-rich diet, abstinence from alcohol and rest, common medication includes vitamin B, vitamin E, vitamin C, etc. But these treatments are less than satisfactory. There remains a need for an effective method for treating liver cirrhosis and other forms of liver disease.

### SUMMARY OF THE INVENTION

The invention provides cell-based methods for the treatment of liver disease in a subject in need thereof. In some aspects, the invention provides therapies for increasing blood flow to the liver in a subject, such as, but not limited to, by promoting the formation of blood vessels. In one aspect, the invention provides therapies comprising the introduction into a patient of cells that can differentiate into endothelial cells or that promote the differentiation of cells from the subject into endothelial cells. Such cells comprise stem cells and progenitor cells. The cells may be isolated from bone marrow, peripheral blood, umbilical cord cells or from other sources.

One aspect of the invention provides a method for improving liver function in a subject in need thereof, comprising administering to said subject a composition comprising AC133+ cells. In certain embodiments, administering of the endothelial precursor cells results in increased neovascularization of said liver.

A related aspect of the invention provides a method of treating liver disease in a subject in need thereof, comprising administering to said subject a composition comprising AC133+ cells. In certain embodiments, administering of the composition to the subject reduces one or more symptoms of the liver disease. In certain embodiments, the reduced symptoms are selected from: loss of liver function, ischemia, fibrosis, cirrhosis.

Another related aspect of the invention provides methods of regenerating liver tissue in a subject in need thereof, comprising administering to said subject a composition comprising AC133+ cells. In certain embodiments, the subject has liver disease. In certain embodiments, the subject is a liver transplant donor or recipient.

In certain embodiments of the prior methods, the subject is afflicted with liver ischemia, liver fibrosis, liver cirrhosis, acute liver failure, Alagille syndrome, alcohol liver disease, Alpha 1—antitrypsin deficiency, autoimmune hepatitis, biliary atresia, chronic hepatitis, cirrhosis, cholestatic liver disease, cystic disease of the liver, fatty liver, galactosemia, gallstones, Gilbert's syndrome, hemochromatosis, hepatitis A, hepatitis B, hepatitis C, liver cancer, neonatal hepatitis, non-alcoholic liver disease, non-alcoholic steatohepatitis, porphyria, primary biliary cirrhosis, primary sclerosing cholangitis, Reye's syndrome, sarcoidosis, steatohepatitis, tyrosinemia, type I glycogen storage disease, viral hepatitis, Wilson's disease, or any combination thereof. In certain embodiments, the subject is afflicted with liver cirrhosis. In certain embodiments, the liver cirrhosis is selected from micronodular, macronodular and mixed cirrhosis.

In certain embodiments of the prior methods, the subject is a liver transplant donor. In certain embodiments of the prior methods, the subject is a mammal. In preferred embodiments of the prior methods, the subject is human.

In certain embodiments of the prior methods, the AC133+ cells are isolated from umbilical cord blood, bone marrow or peripheral blood. In certain embodiments of the prior methods, the AC133+ cells are autologous to the subject. In certain embodiments of the prior methods, the AC133+ cells are allogeneic to the subject.

In certain embodiments of the prior methods, the AC133+ cells are administered by infusion into an artery, via the Edmonton protocol, or via direct application. In certain embodiments, direct application may comprise implanting cells directly to the liver. In certain embodiments, the cells may be embedded in a matrix or gel that is implanted in contact with the liver.

In certain embodiments of the prior methods, the composition comprises a matrix in which the cells are embedded. In certain embodiments of the prior methods, the matrix comprises: polyethylene glycol (PEG), collagen, fibrin, or a combination thereof. In certain embodiments of the prior methods, the fibrin matrix is polymerized from a solution that contains 50 mg/ml to 400 mg/ml fibrinogen and 250 units/mL to 2000 units/mL thrombin.

In certain embodiments of the prior methods, the composition further comprises serum from the subject. In certain embodiments of the prior methods, the composition further comprises soluble human fibronectin, hyaluronan or type I collagen, or a combination thereof.

In certain embodiments of the prior methods, the composition further comprises mesenchymal stem cells. In certain embodiments, mesenchymal stem cells are isolated from umbilical cord blood, bone marrow or peripheral blood. In certain embodiments, the mesenchymal stem cells are isolated from umbilical cord blood. In certain embodiments, the mesenchymal stem cells and the AC133+ cells are mixed prior to administering into the subject.

In certain embodiments of the prior methods, further comprising administering to the subject a diaretic agent, ursodeoxycholic acid, an anti-nausea agent, or a combination thereof. In certain embodiments of the prior methods, the composition further comprises a diaretic agent, ursodeoxycholic acid, an anti-nausea agent, or a combination thereof.

In certain embodiments of the prior methods, the composition further comprises administering to the subject a cytokine, chemokine or growth factor. In certain embodiments, the growth factor is bFGF or VEGF. In certain embodiments, the growth factor is PDGF. In certain embodiments, the growth factor is delivered by injection or is released from a matrix. In certain embodiments, the growth factor is introduced by gene therapy. In certain embodiments of the prior methods, the composition further comprises administering to the subject an anticoagulant.

In certain embodiments of the prior methods, the AC133+ cells are selected from AC133+CD34− cells; AC133+CD34+ cells; or combinations thereof. In certain embodiments, the AC133+ cells are AC133+CD34+KDR-CXCR4− cells. In certain embodiments, at least 10% of cells in the composition are AC133+ cells. In certain embodiments, at least 50% of cells in the composition are AC133+ cells. In certain embodiments, at least 75% of cells in the composition are AC133+ cells.

In certain embodiments of the prior methods, the AC133+ cells are expanded in vitro prior to administering to the subject. In certain embodiments, the expansion in vitro promotes the formation of endothelial cells. In certain embodiments, the expansion in vitro promotes the formation of endothelial cells comprises cell culture media comprising: (a) FBS; (b) horse serum; (c) hydrocortisone; (d) Stem cell growth factor (SCGF); (e) VEGF; or (f) a combination thereof. In certain embodiments of the prior methods, the AC133+ cells are held in a solution comprising buffered saline for 6-36 hours prior to administering to the subject. In certain embodiments of the prior methods, the composition comprises between 1×10⁴ to 5×10⁸ cells.

In certain embodiments of the prior methods, administering of the composition to the subject consist of a single administration of the composition to the subject. In certain embodiments of the prior methods, administering of the composition to the subject consist of multiple administrations of the composition to the subject.

In certain embodiments of the prior methods, administering of the composition reduces, delays or eliminates the need for liver transplantation. In certain embodiments of the prior methods, administering of the composition increases the likelihood of survival for one year by at least 25% to allow for liver transplantation. In certain embodiments of the prior methods, administering of the composition increases the likelihood of survival for one year by at least 30, 35, 40, 45, 50, 55, 60, 65, 70, 75, 80, 85, 90, 95, or 100% to allow for liver transplantation. In certain embodiments of the prior methods, administering of the composition increases the likelihood of survival for two years by at least 25, 30, 35, 40, 45, 50, 55, 60, 65, 70, 75, 80, 85, 90, 95, or 100% to allow for liver transplantation. In certain embodiments of the prior methods, administering of the composition increases the likelihood of survival for three years by at least 25, 30, 35, 40, 45, 50, 55, 60, 65, 70, 75, 80, 85, 90, 95, or 100% to allow for liver transplantation. In certain embodiments of the prior methods, administering of the composition increases the likelihood of survival for four years by at least 25, 30, 35, 40, 45, 50, 55, 60, 65, 70, 75, 80, 85, 90, 95, or 100% to allow for liver transplantation. In certain embodiments of the prior methods, administering of the composition increases the likelihood of survival for five years by at least 25, 30, 35, 40, 45, 50, 55, 60, 65, 70, 75, 80, 85, 90, 95, or 100% to allow for liver transplantation. In certain embodiments of the prior methods, administering of the composition increases the likelihood of survival for ten years by at least 25, 30, 35, 40, 45, 50, 55, 60, 65, 70, 75, 80, 85, 90, 95, or 100% to allow for liver transplantation. In certain embodiments of the prior methods, administering of the composition reduces, delays or eliminates the need for hepatic resection.

One aspect of the invention provides cellular therapeutics to enhance vasculogenesis and collateralization around blocked/narrowed vessels to relieve ischemia in the liver. Clinical use of autologous patient-derived sources of stem cells is advantageous to avoid potential adverse allogeneic immune reactivity.

One aspect of the invention provides an implantable matrix comprising a plurality of AC133+ cells. In certain embodiments, the matrix comprises: polyethylene glycol (PEG), collagen, fibrin, or any combination thereof. In certain embodiments, the fibrin matrix is polymerized from a solution that contains 50 mg/ml to 400 mg/ml fibrinogen and 250 units/mL to 2000 units/mL thrombin.

In certain embodiments, the matrix further comprising mesenchymal stem cells, hepatocytes, hepatocytes progenitor cells, or any combination thereof. In certain embodiments, comprising a recombinant peptide. In certain embodiments, the recombinant peptide is selected from: transforming growth factor, tumor necrosis factor-alpha, basic fibroblast growth factor (bFGF), CX chemokine receptor (CXCR)-4, CXCR-5, vascular endothelial growth factor (VEGF), hepatocyte growth factor (HGF), fibroblast growth factor (FGF), stromal cell-derived factor 1 (SDF-1), angiopoietin-, leukemia inhibitory factor, interleukins IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), FMS-like tyrosine kinase 3 (Flt3)-ligand, B cell activating factor, artemin, bone morphogenic protein factors, epidermal growth factor (EGF), glial derived neurotrophic factor, lymphotactin, macrophage inflammatory proteins, myostatin, neurturin, nerve growth factors, platelet derived growth factors, placental growth factor, pleiotrophin, stem cell factor, or any combination thereof.

One aspect of the invention provides a method for improving liver function comprising increasing serum levels of albumin, platelet counts, or both. In one embodiment, improving liver function comprises increasing serum levels of albumin to at least 2.8 g/dL. In another embodiment, improving liver function comprises increasing serum levels of albumin to at least 3.5 g/dL.

One aspect of the invention provides a method for improving liver function comprising decreasing serum levels of alanine aminotransferase (ALT), aspartate aminotransferase (AST), gamma glutamic transpeptidase (GGT), alkaline phosphatase (ALP), bilirubin, or decreasing the results of a prothrombin time test, or any combination thereof. In one embodiment, improving liver function comprises decreasing serum levels of any one of ALT, AST, GGT, ALP, or bilirubin by at least two-fold.

In another embodiment, improving liver function comprises decreasing serum levels of any one of ALT, AST, GGT, ALP, or bilirubin by at least five-fold. In yet another embodiment, improving liver function comprises decreasing serum levels of any one of ALT, AST, GGT, ALP, or bilirubin by at least ten-fold.

### DETAILED DESCRIPTION OF THE INVENTION

### I. Overview

The invention broadly relates to a cell-based therapy for the treatment of liver disease and/or ischemic tissue. Ischemic tissue may be treated by increasing the blood flow to the tissue. Such increase in blood flow may be mediated, for example, by increasing the number of blood vessels which supply that tissue. The production of blood vessels is accomplished by two main processes: angiogenesis and vasculogenesis. Angiogenesis refers to the production of vascular tissue from fully differentiated endothelial cells derived from pre-existing native blood vessels. Angiogenesis is induced by complex signaling mechanisms of cytokines including vascular endothelial growth factor (VEGF), basic fibroblast growth factor (bFGF), and other mediators. This process is mediated by the encroachment of “activated” endothelial cells through the disrupted basement membrane into the interstitium possibly via an ischemic signal. “Therapeutic angiogenesis” refers to utilizing cytokines derived from gene or recombinant therapy, to induce or augment collateral blood vessel production in patients with ischemic vascular diseases.

In contrast, vasculogenesis, which until recently was believed to occur only in embryos, is the formation of vascular tissues in situ from endothelial precursor cells (EPCs) or angioblasts. Formation of blood islands or clusters of stem cells originating from a common ancestor, the hemangioblast, initiates the process. In these islands or clusters, peripherally located EPCs mature into the endothelium while the centrally located hematopoietic stem cells (HSCs) give rise to blood cells. As used herein, “therapeutic vasculogenesis” refers to neogenesis of vascular tissues by introduction of exogenous endothelial producing cells into the subject cells into a subject.

The invention generally provides methods of increasing blood flow to a liver. More specifically, the invention provides methods for treating a liver in a subject in need thereof, comprising administering to said subject a composition comprising cells. In one embodiment, the cells are enriched human endothelial generating cells and/or human mesenchymal stem cells. As used herein, human endothelial generating cells refers to cells capable of differentiating into human endothelial cells, and include but are not limited to, AC133+ hemagioblasts and endothelial precursor cells. In one embodiment, the compositing comprises cells isolated from umbilical cord blood, such as stem cells.

Another aspect of the invention provides a method for inducing the formation of blood vessels in a liver in a subject in need thereof, comprising administering to the subject a composition comprising cells. In one embodiment, the cells are enriched human endothelial generating cells and/or human mesenchymal stem cells. As used herein, human endothelial generating cells refers to cells capable of differentiating into human endothelial cells, and include but are not limited to, AC133+ hemagioblasts and endothelial precursor cells. In one embodiment, the compositing comprises cells isolated from umbilical cord blood, such as stem cells.

Another aspect of the invention provides a method of treating liver disease in a subject in need thereof, comprising administering to said subject a composition comprising cells. In one embodiment, the cells are enriched human endothelial generating cells and/or human mesenchymal stem cells. As used herein, human endothelial generating cells refers to cells capable of differentiating into human endothelial cells, and include but are not limited to, AC133+ hemagioblasts and endothelial precursor cells. In one embodiment, the compositing comprises cells isolated from umbilical cord blood, such as stem cells. Some examples of liver diseases include liver ischemia, liver fibrosis, liver cirrhosis, acute liver failure, Alagille syndrome, alcohol liver disease, Alpha 1—antitrypsin deficiency, autoimmune hepatitis, biliary atresia, chronic hepatitis, cirrhosis, cholestatic liver disease, cystic disease of the liver, fatty liver, galactosemia, gallstones, Gilbert's syndrome, hemochromatosis, hepatitis A, hepatitis B, hepatitis C, liver cancer, neonatal hepatitis, non-alcoholic liver disease, non-alcoholic steatohepatitis, porphyria, primary biliary cirrhosis, primary sclerosing cholangitis, Reye's syndrome, sarcoidosis, steatohepatitis, tyrosinemia, type I glycogen storage disease, viral hepatitis, Wilson's disease, or any combination thereof. In certain embodiments, the subject is afflicted with liver cirrhosis. In certain embodiments, the liver cirrhosis is selected from micronodular, macronodular and mixed cirrhosis. Additional liver diseases of the application can be found in Harrison's Principles of Internal Medicine 15th Edition, Braunwald et al, 2001, McGraw-Hill, U.S.A., and THE MERCK MANUAL, 17TH Edition, (Beers and Berkow, Ed.), 1999, Whitehouse Station, N.J. incorporated herein by reference.

Yet another aspect of the invention provides a method for improving blood flow to a liver in a subject in need thereof, comprising administering to said subject allogeneic enriched CD133+ cells, such as CD133+/CD34+ cells, isolated from umbilical cord blood. In one embodiment, the CD133+ cells are administered via the Edmonton protocol. In one embodiment, the cells are administered in an amount sufficient to improve blood flow to said liver. One embodiment of this method further comprises administering to said subject human mesenchymal stem cells, either as part of the same composition or as a second composition.

In one preferred embodiment of the methods described herein, the subject is a human. In another embodiment, the subject is a nonhuman mammal. In another embodiment, the subject is an adult, a teenager, a child, a newborn, an embryo or a fetus. In one embodiment, the mammal is a post-partum mammal.

In one embodiment of the methods described herein for treatment of a subject, the cells used in the therapies are isolated from the subject's own umbilical cord blood. Such umbilical cord blood may be cryopreserved at or about the time the subject is born for use when needed. In one embodiment of the methods described herein, the treatment of the ischemic tissue induces formation of blood vessels supplying blood to the ischemic tissue, blood flow to the ischemic tissue, oxygen supply to the ischemic tissue, or a combination thereof. In one embodiment, blood flow to the ischemic tissue is improved by at least 5, 10, 15, 20, 25, 30, 40, 50, 60, 70, 80, 90 or 100% or more by the treatment. In one embodiment, oxygen supply to the ischemic tissue is improved by at least 5, 10, 15, 20, 25, 30, 40, 50, 60, 70, 80, 90 or 100% or more by the treatment.

In one embodiment of the methods described herein, the human endothelial generating cells are human endothelial precursor cells. In one embodiment of the methods described herein, the endothelial generating cells are isolated from bone marrow, from peripheral blood, or more preferably, from umbilical cord blood. In one embodiment, the endothelial generating cells, such as AC133+ hemangioblasts, are culture-expanded under endothelial cell-promoting culture conditions prior to administration to the subject. Such expansion may result in endothelial precursor cells. In another embodiment, the endothelial generating cells are enriched at least 1.5, 2, 3, 4, 5, 10, 15 or 20-fold prior to the prior to administration to the subject. Enrichment can generally be achieved by removing at least some non-endothelial generating cells from a composition comprising both endothelial generating cells and non-endothelial generating cells, by propagating endothelial generating cells under culture conditions which increase their numbers relative to non-endothelial generating cells, or by a combination thereof. In one embodiment of the methods described herein, the endothelial generating cells are hemangioblasts, hematopoetic stem cells, or more preferably endothelial progenitor cells. In specific embodiments of the methods described herein, a combination of these cells are administered to the subject.

In an embodiment of the methods described herein, the endothelial generating cells, such as endothelial precursor cells, are CD31⁺, CD146⁺, CD133⁺, CD34⁺, VE-cadherin⁺ or a combination thereof. In a specific embodiment, the endothelial generating cells are CD133⁺/CD34⁺ cells. In other specific embodiments of the methods described herein, the endothelial generating cells, such as endothelial precursor cells or hemangioblast, are autologous, allogeneic, or HLA-compatible with the subject.

In specific embodiments of the methods described herein, the human mesenchymal stem cells are isolated from bone marrow or from umbilical cord blood, and may be culture-expanded prior their administration to the subject. In a specific embodiment, the mesenchymal stem cells are culture-expanded to enrich for cells containing surface antigens identified by monoclonal antibodies SH2, SH3 or SH4, prior to administering the human mesenchymal stem cells to the subject.

In specific embodiments of the methods described herein, the human mesenchymal stem cells are autologous, allogeneic, or HLA compatible with the subject. The number of endothelial generating cells and/or mesenchymal stem cells administered to an individual afflicted with an ischemic tissue will vary according to the severity of the ischemia, the duration of the ischemic condition, the size of the tissue that is ischemic, and the method of delivery. In one embodiment of the methods described herein, the therapeutically effective amount of enriched human endothelial generating cells and/or enriched human mesenchymal stem cells is a safe and effective amount. In another specific embodiment, the amount of each cell type is at least 1×10⁴ cells. In another embodiment, the amount of enriched human endothelial generating cells and of enriched human mesenchymal stem cells administered to the subject in the methods described herein is between about 10⁴ and about 5×10⁸ cells. In certain embodiments, the number of cells may be between 10⁴ and 5×10⁷, 10⁴ and 5×10⁶, 10⁴ and 5×10⁵, 10⁵ and 5×10⁶, 10⁶ and 5×10⁷, 10⁷ and 5×10⁸, 10⁶ and 5×10⁸, and 10⁵ and 5×10⁸.

When a combination of cell types is administered, both cells types of cells maybe autologous, allogeneic, or HLA compatible with the subject, whereas in other embodiments one cell type may be autologous and the other allogenic or HLA compatible with the subject.

The amount of cells administered to the subject will depend on the mode of administration and the site of administration. For example, a therapeutically effective cell dose via the Edmonton protocol may be lower than that for intra-arterial injection. When both enriched human endothelial generating cells and enriched human mesenchymal stem cells are administered to the subject, the ratio of the two cell types may be, for example, from about 20:1 to about 1:20, from about 10:1 to about 1:10, from about 5:1 to about 1:5, and form about 2:1 to about 1:2, or about 1:1.

In embodiments of the methods described herein, administering to the subject is effected via an infusion of cells into the subject. The infusion may comprise a systemic infusion of cells into the subject, or it may comprise an infusion of cells in the proximity of the ischemic tissue, so as to facilitate the migration of cells to the ischemic tissue, or it may comprise both. The infusion may also be performed on the blood vessels that supply blood to the ischemic tissue, such as the portal vein or a hepatic artery, or to blood vessels which remove blood from the ischemic tissue, such as a hepatic vein. In specific embodiments of the methods described herein, the infusion of cells into the subject comprises an infusion into bone marrow, an intra-arterial infusion, an intramuscular infusion, an intravenous infusion or an intradermal infusion. In one embodiment of the methods described herein, the cells are administered to the subject by infusion into at least one artery.

In some embodiments of the methods described herein, administration of the cells to the subject is performed using an intra-arterial catheter, such as but not limited to a balloon catheter, or by using a stent. Any method currently available for delivering cells to a subject may be used to administer cells to a subject in the methods described herein.

In some embodiments of the methods described herein, at least one recombinant polypeptide or at least one drug is further administered to the subject. In one embodiment, the recombinant polypeptide comprises a growth factor, a chemokine, a cytokine, or a receptor of a growth factor, a chemokine, or a cytokine. In preferred embodiments, the recombinant polypeptide promotes angiogenesis, vasculogenesis, or both. In some embodiments, the recombinant polypeptide promotes the proliferation, the differentiation or the ability of the endothelial generating cells or the mesenchymal stem cells to localize to the ischemic tissue or to interact with cells from the ischemic tissue. In specific embodiments, the recombinant polypeptide comprises VEGF, BFGF, SDF, CXCR-4, CXCR-5 or any combination thereof.

In some embodiments of the methods described herein, at least one treatment is further administered to the subject. In one embodiment, the treatment comprises vegetable protein-rich diet, abstinence from alcohol, bed rest, vitamin B, vitamin E, or vitamin C or any combination thereof. In preferred embodiments, the treatment promotes decreased need for liver transplant or resection or increased survival.

In some embodiments of the methods described herein, the endothelial generating cells, such as AC133+ hemangioblasts, or the mesenchymal stem cells, or both, are genetically modified. In a specific embodiment, the cells are genetically modified to express a recombinant polypeptide. In one embodiment the recombinant polypeptide is a growth factor, chemokine or cytokine, or a receptor for growth factors, chemokines or cytokines. In another specific embodiment, the recombinant polypeptide is VEGF, bFGF, SDF, CXCR-4 or CXCR-5. In another embodiment, the recombinant polypeptide expressed by the genetically modified cells promotes the proliferation, the differentiation or the ability of the endothelial generating cells or the mesenchymal stem cells to localize to the ischemic tissue. In another embodiments, the genetic modification enhances the ability of the modified cells to interact with cells at the site of the ischemic tissue. In a related embodiment, the endothelial generating cells, such as endothelial progenitor cells, or the mesenchymal stem cells, or both, are non-genetically modified, such as with polypeptides, antibodies, or antibody binding proteins, prior to administration to the patient. In some embodiments, this treatment is intended to increase the localization of the modified cells to the ischemic tissue.

In some embodiments of the methods described herein, the endothelial generating cells are hemangioblasts cells. In one embodiment, the hemangioblasts are CD133+ cells, CD34+ cells, or more preferably CD133+/CD34+ cells. In embodiments of the methods described herein, the endothelial generating cells are expanded in culture prior to administration to the subject. In specific embodiments, the endothelial generating cells are culture-expanded under endothelial cell-promoting culture conditions prior to administration to the subject.

In some preferred embodiments of the methods described herein, the endothelial generating cells and the mesenchymal stem cells, are enriched prior to administration. By enrichment it is meant that the concentration of the cells relative to that of other cells is increased. Enrichment may be accomplished by removing other types of cells from the composition containing these cells, by culturing the cells under conditions which improve their proliferation over those of other cells, or by any method known in the art for enriching one cell type over another. In some embodiments, the cells used in the methods described herein are enriched at least about two-fold, about five-fold, about twenty-fold, about fifty-fold, about one hundred-fold, about five hundred-fold, about one thousand-fold, about five thousand-fold, about ten thousand-fold, or by about fifty thousand fold.

One aspect of the invention provides a composition for the treatment of ischemia in a subject, comprising a population of cells wherein at least 50% of the cells express CD133+, wherein the CD133+ cells are derived from umbilical cord blood, bone marrow, or peripheral blood, and wherein the CD133+ cells can differentiate into hematopoetic and endothelial cell lineages; and at least an additional component. In a preferred embodiment, the CD133+ cells are derived from cryopreserved and thawed umbilical cord blood.

In some embodiments, the additional component is human serum, preferably human serum from the subject for which administrations of the cells is intended. In another embodiment, the additional component may comprise a component of human serum, such as human serum albumin. In another embodiment, the second component comprises a preservative, such as citrate phosphate dextrose adenine (CPDA) or heparin. In another embodiment, the second component is soluble human fibronectin. In a specific embodiment, the soluble human fibronectin is found a concentration of at least 1 ng/mL, or mote preferably more than 10 ng/ml. In another embodiment, the cell viability of the CD133+ cells is at least 10% greater in the presence of the fibronectin than in its absence.

In some embodiments of the compositions provided herein, the compositions are provided frozen or cryopreserved. In other embodiments, the composition comprises a desicated population of cells. One specific aspect of the invention provides a composition for the treatment of ischemia in a subject, comprising (i) a desiccated population of cells wherein at least 50% of the cells express CD133+, wherein the CD133+ cells are derived from cryopreserved and thawed human umbilical cord blood and wherein the CD133+ cells can differentiate into hematopoetic and endothelial cell lineages; and (ii) at least one carbohydrate, such as trehalose. The tetrahalose may be present at a concentration of at least 25 nM. Methods for generating desiccated cell populations is described, for example, in U.S. Pat. No. 6,528,309.

In some embodiments of the methods described herein, the cells which are to be administered to the subject are incubated in a buffer, such as a saline buffer. In one preferred embodiment, the buffer comprises human blood serum isolated from the same subject who is the recipient of the therapy. Human serum may be isolated using standard procedures. A solution comprising human blood serum may also be used to thaw a sample of cells that has been cryopreserved. In some embodiments, the solution comprising human serum comprises between 1-20% human serum, or more preferably 5-15%.

Some aspect of the embodiments provides methods for inducing neovascularization in a liver in a subject in need thereof. There are numerous conditions that cause the necessity of a mammal to be in need of liver neovascularization. For example, the mammal may have had a liver transplant, liver ischemia, liver fibrosis, liver cirrhosis, acute liver failure, Alagille syndrome, alcohol liver disease, Alpha 1-antitrypsin deficiency, autoimmune hepatitis, biliary atresia, chronic hepatitis, cirrhosis, cholestatic liver disease, cystic disease of the liver, fatty liver, galactosemia, gallstones, Gilbert's syndrome, hemochromatosis, hepatitis A, hepatitis B, hepatitis C, liver cancer, neonatal hepatitis, non-alcoholic liver disease, non-alcoholic steatohepatitis, porphyria, primary biliary cirrhosis, primary sclerosing cholangitis, Reye's syndrome, sarcoidosis, steatohepatitis, tyrosinemia, type I glycogen storage disease, viral hepatitis, Wilson's disease, or any combination thereof.

The cells of the present invention may be recruited into the site that requires neovascularization. For example, stem cells may be mobilized (i.e., recruited) into the circulating peripheral blood by means of cytokines, such as, for example, G-CSF, GM-CSF, VEGF, SCF (c-kit ligand) and bFGF, chemokines, such as SDF-1, or Interleukins, such as interleukins 1 and 8. Stem cells may also be recruited to the circulating peripheral blood of a mammal if the mammal sustains, or is caused to sustain, an injury.

Another aspect of the invention provides a method for improving blood flow to a peripheral arterial vascular bed having an area of ischemic but viable tissue in a subject in need thereof, comprising administering to said subject a therapeutically effective amount of enriched CD133⁺/CD34⁺ cells isolated from umbilical cord blood, bone marrow or peripheral blood, wherein the enriched CD133⁺/CD34⁺ cells are administered by catheter infusion into at least one artery that provides collateral flow to said tissue, and wherein administering of the CD133⁺/CD34⁺ cells results in improved blood flow to said ischemic tissue. In a specific embodiment, the subject is afflicted with liver disease.

Another aspect of the invention also provides a method for improving blood flow to a liver in a subject in need thereof, comprising administering to said subject a therapeutically effective amount of enriched CD133⁺/CD34⁺ cells isolated from umbilical cord blood, bone marrow or peripheral blood, wherein the enriched CD133⁺/CD34⁺ cells are administered by the Edmonton protocol, wherein administering of the CD133⁺/CD34⁺ cells results in improved blood flow to said liver. In a specific embodiment, the CD133+/CD34+ cells are isolated from umbilical cord blood.

An additional aspect of the invention provides a method for improving blood flow to a liver in a subject in need thereof, comprising administering to said subject a therapeutically effective amount of a composition comprising (i) enriched CD133⁺ cells isolated from umbilical cord blood; and (ii) serum from the subject; wherein the enriched CD133⁺ cells are administered by the Edmonton protocol, and wherein administering of the CD133⁺ cells results in improved blood flow to said liver.

One aspect of the invention provides a method for improving blood flow to a liver in a subject in need thereof, comprising (i) isolating bone marrow from the subject; (ii) selecting CD133+ cells from the bone marrow to generate an enriched population of CD133+ cells; (iii) holding the enriched population of CD133+ cells in a solution comprising buffered saline for 6-36 hours; (iv) administering the enriched population of CD133+ cells to the subject by the Edmonton protocol, thereby improving blood flow to said liver. In a specific embodiment, the solution further comprises serum from the subject, such as serum derived from peripheral cord blood or from bone marrow blood. In another embodiment, the solution further comprises soluble human fibronectin, hyaluronan or type I collagen, or a combination thereof.

In another embodiment of the methods for improving blood flow to a liver in a subject in need thereof, step (iv) further comprises administering to the subject mesenchymal stem cells (MSCs) isolated from the subject's bone marrow, or more preferably, from umbilical cord blood. In some embodiments, the MSCs are administered by the Edmonton protocol. The MSCs be mixed with the CD133+ cells are mixed prior to administration into the subject, or they may be injected separately.

In another embodiment of the methods for improving blood flow to a liver, or for treating liver disease, in a subject in need thereof, at least one a cytokine, chemokine or growth factor is administered to the subject. Exemplary growth factors include bFGF or VEGF. In preferred embodiments, the cytokine, chemokine or growth factor promotes angiogenesis. Other embodiments further comprise administering to the subject an anticoagulant.

In another embodiment of the methods described herein for improving blood flow to a liver in a subject in need thereof, the CD133+ cells are CD133+CD34+KDR-CXCR4− cells. In some embodiments, at least 10%, 20%, 30%, 40%, 50%, 60%, 70%, 75%, 80%, 90%, 95%, 98%, 99% of cells in the enriched population are CD133+ cells. In a specific embodiments, the enriched population of CD133+ also contains the following percentages of cells having specific markers: CD133+ (60%-99%), CD34+ (75%-99%), KDR (EGFR2) (0-10%), CD105 (15%-30%) and CXCR4 (2%-15%).

In one embodiment of the foregoing methods, the enriched population of CD133+ cells is not expanded in culture prior to administration into the subject. In one embodiment, the therapeutic cells have undergone less than 5, 4, 3, 2 or 1 cell divisions between their isolation from bone marrow, umbilical cord bllod, or peripheral blood, and their administration to the subject. Alternatively, in one embodiment, after step (ii) and before step (iii), the enriched population of CD133+ cells is expanded in vitro under conditions that promote the formation of endothelial cells. In one embodiment, the conditions that promote the formation of endothelial cells comprise cell culture media comprising (a) FBS; (b) horse serum; (c) hydrocortisone; (d) stem cell growth factor (SCGF); (e) VEGF; or (f) a combination thereof. In one specific embodiment, the conditions that promote the formation of endothelial cells comprise cell culture media comprising (a) 5-15% FBS; (b) 5-15% horse serum; (c) 0.1-10 mM hydrocortisone; (d) 10-1000 ng/ml of stem cell growth factor (SCGF); (e) 5-500 ng/ml of VEGF; (f) or a combination thereof. In an exemplary embodiment, the conditions that promote the formation of endothelial cells comprise cell culture media comprising (a) 10% FBS; (b) 10% horse serum; (c) 1 mM hydrocortisone; (d) 100 ng/ml of stem cell growth factor (SCGF); (e) 50 ng/ml of VEGF; or (f) a combination thereof.

In one embodiment of the foregoing methods, the therapeutically effective amount of CD133+ cells comprises between 1×10⁴ to 5×10⁸ cells. In another embodiment, the therapeutically effective amount of the CD133+ cells is the minimum number of cells necessary for increased blood flow induction to the liver.

### II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The terms “CD133” and “AC133” are used interchangeably and refer to the antigen as described in Yin et al. 1997. Blood 90(12):5002-12.

### III. Human Endothelial Generating Cells

The methods described herein comprise the use of endothelial generating cells (EGCs). ECGs comprise any cell which can differentiate into an endothelial cell. EGCs are preferably mammalian, primate or human cells. ECGs comprise embryonic stem cells, hemangioblasts, pluripotent stem cells, adult stem cell, hematopoietic stem cells and endothelial precursor cells. In some embodiments of the methods described herein, the endothelial generating cells, such as endothelial precursor cells, are generated in culture from hematopoetic stem cells, CD133+ hemangioblasts or embryonic stem cells.

In a preferred embodiment of the methods for therapeutic neovascularization of liver tissues described herein, the endothelial generating cells are endothelial precursor cells. In a preferred embodiment, the exogenous EPC cells are enriched for CD133⁺ cells. The cell surface marker CD133⁺ is also known as AC133. AC133 is a recently discovered marker for HSCs from peripheral blood, bone marrow, fetal liver and umbilical cord blood (Gehling et al., 2000, Blood. 95(10): 3106-12; Yin et al. 1997. Blood 90(12):5002-12; Buhring et al. 1999. Ann NY Acad SCI 99 872: 25-39; Majka et al. 2000. Folia Histochem Cytobiol. 38:53-63, U.S. Patent Publication Nos. 2004/0258670, 2005/0069527, the entire teachings of which are herein incorporated by reference). Antibodies which recognize the CD133 antigen are described in U.S. Pat. No. 5,843,633. In another embodiment, EGCs are CD34⁺ cells. In yet another embodiment, the EGCs are CD133⁺/CD34⁺ cells. AC133⁺ hematopoietic stem cells are particularly useful in the methods described herein, as these cells have been shown to differentiate into endothelial cells after short-term culturing.

Bone marrow, peripheral blood or umbilical cord blood (UCB) are potential sources of therapeutic CD133⁺ cells. Accordingly, the EGCs used in the methods described herein may be isolated from any of these three sources. A simple isolation technique, the collection of adherent cells after four days of culture of fresh UCB, produces a cell population with significant proliferative and colony forming potential as previously described (Mandel, D. et al. Blood 98 (11), 55b. (2001), the contents of which are hereby incorporated by reference in their entirety. In one embodiment, the cells used in the therapeutic methods described herein are not isolated from bone marrow. In one embodiment, the cells used in the therapeutic methods described herein are not isolated from peripheral blood. In one embodiment, the cells used in the therapeutic methods described herein are not isolated from umbilical blood.

The data described in the Exemplification section supports morphological features of UCB-derived EPCs consistent with vascular endothelial cultures. After short-term culture in media designed to expand vascular endothelial cells, many of these cultured cells exhibit surface markers that are considered specific to endothelial cells including CD31 and CD146 (P1H12). Accordingly, in a preferred embodiment, the EPCs used in the methods described herein give rise to endothelial cells which express CD31 and CD146 (P1H12) after short-term culture in media designed to expand vascular endothelial cells. The majority of the cells derived from EPCs using the methods described herein endocytose acLDL and a minority exhibit lectin binding, two important cytochemical endothelial characteristics. In addition, culture expanded UCB EPC produce von Willebrand Factor (vWF).

In one embodiment, the EGCs are isolated from UCB. The use of UCB as an EGC source is advantageous due to its high content of early CD133⁺ stem cells, as well as its robust proliferative capacity of these cells, low immunogenicity, low infectious contamination (including virions), and “off the shelf” clinical application potential with diverse representation of histocompatibility genotypes in banked unrelated UCB. CD133⁺ cells can be positively selected from isolated mononuclear cells from any of the foregoing sources by any method that produces an enriched population of CD133⁺ cells. Several techniques are well known for the rapid isolation of CD133⁺ cells such as, but not limited to, leucopheresis, density gradient fractionation, immunoselection, differential adhesion separation, and the like. As a non-limiting example, MNC can be obtained by density gradient centrifugation and labeled with magnetic bead-conjugated anti-CD133 antibody and passed through one or more magnetic columns to yield positively selected CD133⁺ cells. Additionally or alternatively, MNC can be labeled with a fluorescent antibody to CD133 and sorted by a fluorescence activated cell sorter (FACS) to obtain CD133⁺ cells. Yields and purity of the obtained CD133⁺ cells can vary, depending on the source and the methods used to purifying the cells. Purity obtained after one passage of labeled cells through a magnetic column can be, for example, 75%-85% and, after subsequent FACS, the purity can be increased to 95%-99%.

CD133+ cells may further be purified based on the expression of an additional cell surface molecule, such as CD34. For example, human CD133+ stem cells may be further purified by means of an anti-CD34 antibody, such as the anti-My-10 monoclonal antibody described by Civin in U.S. Pat. No. 5,130,144. The hybridoma cell line that expresses the anti-My monoclonal antibody is available from the American Type Culture Collection, 12301 Parklawn Drive, Rockville, Md. 20852, USA. Some additional sources of antibodies capable of selecting CD34+ cells include AMAC, Westbrook, Me.; Coulter, Hialea, Fla.; and Becton Dickinson, Mountain View, Calif. CD34+ cells may also be isolated by means of comparable antibodies, which may be produced by methods known in the art, such as those described by Civin in U.S. Pat. No. 5,130,144.

CD133+ cells may be further purified by negative selection i.e. removing cells which express a given cell marker. For example, cells expressing CD1, CD2, CD3, CD4, CD5, CD8, CD10, CD11b, CD13, CD14, CD15, CD16, CD19, CD20, CD24, CD25, CD28, CD29, CD33, CD36, CD38, CD41, CD41, CD56, CD66b, CD66e, CD69 or glycophorin A may be negatively selected. In one embodiment, a negative selection is performed for cells expressing CXCR4 or KDR (EFGR2).

The CD133⁺ cells can be allogeneic, autologous or HLA-compatible with the recipient.

The selected CD133⁺ cells can be culture-expanded under endothelial cell-promoting culture conditions prior to the administering step. Alternatively, MNC from bone marrow, peripheral blood or umbilical cord blood can be cultured under short-term culture (e.g., about 24 hours) in endothelial cell-promoting culture conditions, and CD133⁺ cells selected during culture by selection techniques such as those described above.

Several culture media suitable for promoting endothelial cell differentiation are known. As a non-limiting example, one such suitable medium, described in Kalka et al. (2000) PNAS 97: 3422-3427, is EC basal medium-2 (EBM-2) (Clonetics, San Diego) with 5% fetal bovine serum (FBS) and standard SingleQuot™ additives that include human VEGF-1, human basic fibroblast growth factor-2 (FGF), insulin-like growth factor-1 (IGF-1), hydrocortisone, ascorbic acid and heparin.

Additional methods and sources of isolating CD133+ cells are described, for example, in International PCT Application Nos. WO03/095631, WO99/37751, and WO01/94420, and U.S. Patent Publication Nos. 2003/0091547, 2003/0199464 and 2002/0051762, the entire teachings of which are herein incorporated by reference.

On one embodiment of the methods described herein, the EGCs are genetically modified prior to administration to the subject. In one embodiment, EGCs are genetically modified to express a recombinant polypeptide, such as a growth factor, chemokine, or cytokine, or a receptor thereof. In another embodiment, the recombinant peptide is VEGF, BFGF, SDF, CXCR-4 or CXCR-5. In another embodiment, the genetic modification promotes angiogenesis, vasculogenesis, or both. EGCs may be modified, for example, using the methods commonly known in the art, such as by transfection, transformation or transduction, using recombinant expression vectors. The vector may be integrated into chromosomal DNA or be carried as a resident plasmid by the genetically modified ECG. In some embodiments, retroviruses are used to genetically modify the EGCs. Additional genes that may be introduced into the EGCs are described in International PCT Publication No. WO99/37751.

In some embodiments of the methods described herein, the endothelial generating cells comprise CD133+ cells. In specific embodiments of the compositions described herein which comprise CD133+ cells, at least 10% of the cells in the composition are CD133+ cells. In other specific embodiments, at least at least 15%, 20%, 25%, 30%, 35%, 40%, 45%, 50%, 55%, 60%, 65%, 70%, 75%, 80%, 85%, 90%, 95%, 96%, 97%, 98% or 99% of the cells in the composition that is administered to the subject are CD133+ cells.

In one embodiment of the methods described herein, the EGCs do not comprise cells capable of differentiating into hepatocytes. In one embodiment, the cells administered to the subject do not include hepatocytes or hepatocytes progenitor cells.

In one embodiment, the therapeutic methods described herein comprise administering to the subject both EGCs and (i) hepatocytes; (ii) or hepatocytes progenitor cells. Hepatocyte precursor cells are cells capable of differentiating into hepatocytes. Hepatocyte progenitor cells have been described, for example, in Santoni-Rugiu et al. APMIS. 2005 November-December; 113(11-12):876-902; Fausto et al. Hepatology. 2006 February; 43(2 Suppl 1):S45-53 . . . .

### IV. Human Mesenchymal Stem Cells/Stromal Cells

One aspect of the invention provides methods for treating liver disorder in a subject in need thereof, comprising administering to said subject a therapeutically effective amount of enriched human endothelial generating cells and enriched human mesenchymal stem cells. Mesenchymal stem cells are the formative pluripotent blast cells found in the bone marrow and peripheral blood that are capable of differentiating into any of the specific types of connective tissues (i.e., the tissues of the adipose, areolar, osseous, cartilaginous, elastic, and fibrous connective tissues) depending upon various environmental influences. Mesenchymal stem cells are also commonly referred to as “marrow stromal cells” or just “stromal cells”. Mesenchymal progenitor cells, are derived from mesenchymal stem cells and have a more limited differentiating potential, but are able to differentiate into at least two tissues (see for example, FIG. 1 of Minguell et al. 2001 Exp Biol Med (Maywood); 226(6):507-20). As used herein, the term “mesenchymal stem cells” comprises mesenchymal stem cells, mesenchymal progenitor cells and marrow stromal cells.

Applicants have previously reported extensive research on methods to isolate, culture-expand and phenotypically characterize hMSCs, as well as their multi-lineage developmental potential and capacity to regulate a variety of other developmental events including angiogenesis (Fleming, J E Jr. et al. Dev. Dyn. 212, 119-132 (1998); Barry F P et al. Biochem. Biophys. Res. Commun. 265, 134-139 (1999)). Although hMSCs are rare, comprising about 0.01-0.0001% of the total nucleated cells of bone marrow, Applicants have perfected a cell culture methodology for their isolation from bone marrow, purification to homogeneity from other bone marrow cells and mitotic expansion in culture without loss of their stem cell potential (Haynesworth S E et al. Bone 13, 81-88 (1992)). Human adult MSC, although marrow-derived, do not express CD34 or CD45, but have been shown to express IL-6, -7, -8, -11, -12, -14, -15, M-CSF, flt-3 ligand (FL), and SCF in steady state, and do not express IL-3 and TGFβ. Exposure to dexamethasone results in decreased expression of LIF, IL-6 and IL-11 (Haynesworth S E et al. J. Cell Physiol. 166, 585-592 (1996)). Moreover, adhesion molecules expressed by stromal cells of importance in supporting early hemangioblasts, include fibulin-1 and fibulin-2, tenascin-C, stromal cell-derived factor 1 (SDF-1), and collagen type VI.

While not being bound by theory, it is believe that hMSCs home to sites of vascular injury and augment vasculogenesis in concert with early hemangioblasts, via secreted soluble factors and direct cell contact effects. Mesenchymal cells are known to constitutively secrete extracellular matrix-degrading enzymes, primarily matrix metalloproteinase 9, which promote endothelial cell invasion. In addition, mesenchymal cells secrete several pro angiogenic factors including VEGF, bFGF, IL-8, PDGF, and hematopoietic growth factors that promote endothelial cell migration, proliferation, and/or tube formation.

Mesenchymal stem cells for use in the methods according to the invention can be isolated from peripheral blood or bone marrow. A method for preparing hMSC has been described in U.S. Pat. No. 5,486,359. Furthermore, mesenchymal stem cells may also be isolated from umbilical cord blood, as described by Erices et al. 2000 Br. J Haematol 109(1):235-42. In a preferred embodiment of the methods described herein, when the mesenchymal stem cells are isolated from bone marrow or peripheral blood of the subject afflicted with ischemic tissue who will be the recipient of the treatment.

Several techniques are known for the rapid isolation of mesenchymal stem cells including, but are not limited to, leucopheresis, density gradient fractionation, immunoselection, differential adhesion separation, and the like. For example, immunoselection can include isolation of a population of hMSCs using monoclonal antibodies raised against surface antigens expressed by bone marrow-derived hMSCs, i.e., SH2, SH3 or SH4, as described, for example, in U.S. Pat. No. 6,387,367. The SH2 antibody binds to endoglin (CD105), while SH3 and SH4 bind CD73. Further, these monoclonal antibodies provide effective probes which can be utilized for identifying, quantifying and purifying hMSC, regardless of their source in the body. In one embodiment of the methods described herein, mesenchymal stem cells are culture expanded to enrich for cells expressing CD45, CD73, CD105, stro-1, or a combination thereof. In another embodiment, human mesenchymal stem cells are culture-expanded to enrich for cells containing surface antigens identified by monoclonal antibodies SH2, SH3 or SH4, prior to administering the human mesenchymal stem cells to the subject. A stro-1 antibody is described in Gronthos et al., 1996, J. Hematother. 5: 15-23. Further cell surface markers that may be used to enrich for human mesenchymal stem cells, such as those found in Table I, page 237 of Fibbe et al., 2003. Ann. N.Y. Acad. Sci. 996: 235-244.

The hMSC for use in the methods according to the invention can be maintained in culture media which can be chemically defined serum free media or can be a “complete medium”, such as Dulbecco's Modified Eagles Medium supplemented with 10% serum (DMEM). Suitable chemically defined serum free media are described in U.S. Pat. No. 5,908,782 and WO96/39487, and complete media are described in U.S. Pat. No. 5,486,359. Chemically defined medium comprises a minimum essential medium such as Iscove's Modified Dulbecco's Medium (IMDM), supplemented with human serum albumin, human Ex Cyte lipoprotein, transferrin, insulin, vitamins, essential and non-essential amino acides, sodium pyruvate, glutamine and a mitogen. These media stimulate mesenchymal stem cell growth without differentiation. Culture for about 2 weeks results in 10 to 14 doublings of the population of adherent cells. After plating the cells, removal of non-adherent cells by changes of medium every 3 to 4 days results in a highly purified culture of adherent cells that have retained their stem cell characteristics, and can be identified and quantified by their expression of cell surface antigens identified by monoclonal antibodies SH2, SH3 and/or SH4.

On one embodiment of the methods described herein, the mesenchymal stem cells are genetically modified prior to administration to the subject. In one embodiment, the mesenchymal cells are genetically modified to express a recombinant polypeptide, such as a growth factor, chemokine, or cytokine, or a receptor which binds growth factors, chemokines, or cytokines. In another embodiment, the recombinant peptide is vascular endothelial growth factor (VEGF), hepatocyte growth factor (HGF), fibroblast growth factor (FGF), stromal cell-derived factor 1 (SDF-1), or interleukin 8 (IL-8). Mesenchymal stem cells may be modified, for example, using the methods disclosed in U.S. Pat. No. 5,591,625 or the methods described above for EGCs. In another embodiment, the genetic modification promotes angiogenesis, vasculogenesis, or both. The recombinant polypeptide may be, for example, VEGF or angiopoietin-1. U.S. Patent Publication No. 2003/0148952 describes the use of angiopoietin-1 to recruit endothelial precursor cells. In another embodiment, the recombinant polypeptide is selected from the group consisting of leukemia inhibitory factor, IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, B cell activating factor, artemin, bone morphogenic protein factors, epidermal growth factor (EGF), glial derived neurotrophic factor, lymphotactin, macrophage inflammatory proteins, myostatin, neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors, FGF-acidic and basic fibroblast growth factor.

In another embodiment of the methods described herein, the mesenchymal stem cells are modified prior to implantation into the patient so as to promote their targeting to the ischemic tissue. In a specific embodiment, the cells are coated with protein G and with an antibody which binds an antigen that is abundant in sites of ischemic injury.

### V. Methods of Administration

In the methods described herein, the therapeutically effective amount of the endothelial generating cells, such as CD133+ cells, and the therapeutically effective amount hMSCs, can range from the maximum number of cells that is safely received by the subject to the minimum number of cells necessary for either induction of new blood vessel formation in the ischemic tissue or for increasing blood flow to the ischemic tissue. Generally, the therapeutically effective amount of each endothelial generating cells and hMSCs is at least 1×10⁴ per kg of body weight of the subject and, most generally, need not be more than 7×10⁵ of each type of cell per kg. The ratio of CD133+ cells to hMSCs can vary from about 5:1 to about 1:5. A ratio of about 1:1 is preferable. Although it is preferable that the hMSCs are autologous or HLA-compatible with the subject, the hMSCs can be isolated from other individuals or species or from genetically-engineered inbred donor strains, or from in vitro cell cultures.

The therapeutically effective amount of the CD133+ cells and/or the MSCs can be suspended in a pharmaceutically acceptable carrier or excipient. Such a carrier includes but is not limited to basal culture medium plus 1% serum albumin, saline, buffered saline, dextrose, water, and combinations thereof. The formulation should suit the mode of administration. Accordingly, the invention provides a use of human endothelial producing cells, such as CD133+ cells, for the manufacture of a medicament to treat an ischemic tissue in a subject in need thereof. In some embodiments, the medicament further comprises recombinant polypeptides, such as growth factors, chemokines or cytokines. In further embodiments, the medicaments comprise hMSCs. The cells used to manufacture the medicaments may be isolated, derived, or enriched using any of the variations provided for the methods described herein.

In a preferred embodiment, the endothelial generating cell, CD133+ cell and/or the hMSC preparation or composition is formulated in accordance with routine procedures as a pharmaceutical composition adapted for intravenous administration to human beings. Typically, compositions for intravenous, intra-arterial or intracardiac administration are solutions in sterile isotonic aqueous buffer. Where necessary, the composition may also include a local anesthetic to ameliorate any pain at the site of the injection. Generally, the ingredients are supplied either separately or mixed together in unit dosage form, for example, as a cryopreserved concentrate in a hermetically sealed container such as an ampoule indicating the quantity of active agent. When the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

A variety of means for administering cells to subjects will, in view of this specification, be apparent to those of skill in the art. Such methods include injection of the cells into a target site in a subject. Cells may be inserted into a delivery device which facilitates introduction by injection, implantation, or contact with the subjects. Such delivery devices may include tubes, e.g., catheters, for injecting cells and fluids into the body of a recipient subject. In a preferred embodiment, the tubes additionally have a needle, e.g., a syringe, through which the cells of the invention can be introduced into the subject at a desired location. In a preferred embodiment, cells are formulated for administration into a blood vessel via a catheter (where the term “catheter” is intended to include any of the various tube-like systems for delivery of substances to a blood vessel). The cells may be prepared for delivery in a variety of different forms. For example, the cells may be suspended in a solution or gel. Cells may be mixed with a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid, and will often be isotonic. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

Modes of administration of the endothelial generating cells, such as the CD133+ cells, and the hMSCs include but are not limited to intravenous or intra-arterial injection and injection directly into the tissue at the intended site of activity. The preparation can be administered by any convenient route, for example by infusion or bolus injection and can be administered together with other biologically active agents. Administration is preferably systemic. Most preferably, the site of administration is close to or nearest the intended site of activity. In cases when a subject suffers from global ischemia, a systemic administration, such as intravenous administration, is preferred. Without intending to be bound by mechanism, endothelial generating cells such as CD133+ cells and the hMSCs will, when administered, migrate or home to the ischemic tissue in response to chemotactic factors produced due to the injury.

In a preferred embodiment, the endothelial generating cell, CD133⁺ cells and/or the hMSC preparation or composition is delivered by the Edmonton protocol (See for example, Ryan et al., Diabetes. 2001 April; 50(4):710-9, herein incorporated by reference). The cell administrations may be carried out under local anesthetic. A very thin needle may be used, in conjunction with x-rays or other imaging techniques, to locate the main blood vessel in the liver (the portal vein), into which the blood-type-matched islet cells are injected. Once into the liver the EGCs are expected to induce neovascularization. Immunosuppression following the procedure may be desirable. In some embodiments, immunosuppression may be achieved by basiliximab induction therapy, sirolimus and tacrolimus maintenance therapy, aspirin and enoxaparin thromboprophylaxis, pneumocystis carinii prophylaxis with sulfamethoxazole/trimethoprim for 6 months and cytomegalovirus prophylaxis for 3 months if indicated, or by other methods available to a clinician.

The general Edmonton protocol may be varied by one skilled in the art depending on the particular embodiment. For example, immunosuppressants might be omitted with the cells being administered are autologous to the subject. In one exemplary embodiment, allogeneic cells are used without a concurrent administration of immunosuppressants.

In certain embodiments, liver function tests (LFTs) are performed to assess the health of the liver before, during and/or after the methods of the application. LFTs comprise several blood tests indicative of liver function. LFTs that may be used in the invention include, for example, measurements of albumin, various liver enzymes; alanine aminotransferase (ALT), aspartate aminotransferase (AST), gamma glutamic transpeptidase (GGT), and alkaline phosphatase (ALP), bilirubin, and prothrombin time (PT). All of these tests can be performed from a single blood sample or from multiple samples. Additional liver functional tests can be found in Harrison's Principles of Internal Medicine 15th Edition, Braunwald et al, 2001, McGraw-Hill, U.S.A., and THE MERCK MANUAL, 17TH Edition, (Beers and Berkow, Ed.), 1999, Whitehouse Station, N.J. incorporated herein by reference. In certain embodiments, additional markers of hepatocyte function may be examined such as albumin, Alfa-fetoprotein (AFP) and cytochrome P-450.

In certain embodiments, an LFT or a combination of LFTs are performed up to one day, one week, one month, two months, three months, four months, five months, or six months following treatment. In certain embodiments, an LFT or a combination of LFTs are performed during each follow-up exam up to a clinical endpoint.

ALT (alanine aminotransferase) was previously called SGPT is specific for liver damage. The ALT is an enzyme that is produced in the liver cells (hepatocytes) therefore it is more specific for liver disease than some of the other enzymes. It is generally increased in situations where there is damage to the liver cell membranes. All types of liver inflammation can cause raised ALT. Liver inflammation can be caused by fatty infiltration some drugs/medications, alcohol, liver and bile duct disease.

AST (aspartate aminotransferase) was previously called SGOT. This is a mitochondrial enzyme that is also present in heart, muscle, kidney and brain therefore it is less specific for liver disease. In many cases of liver inflammation, the ALT and AST activities are elevated roughly in a 1:1 ratio.

ALP (alkaline phosphatase) is elevated in many types of liver disease but also in non-liver related diseases. Alkaline phosphatase is an enzyme, or more precisely a family of related enzymes, that is produced in the bile ducts and sinusoidal membranes of the liver but is also present in many other tissues. An elevation in the level of serum alkaline phosphatase is raised in bile duct blockage from any cause. Therefore raised ALP in isolation will generally lead a physician to further investigate this area. Conditions such as Primary Biliary Cirrhosis and Sclerosing Cholangitis will generally show a raised ALP. Raised levels may also occur in cirrhosis and liver cancer. Alkaline phosphatase is also produced in bone and blood activity can also be increased in some bone disorders.

GGT (gamma glutamyl transpeptidase) is often elevated in those who use alcohol or other liver toxic substances to excess. An enzyme produced in many tissues as well as the liver. Like alkaline phosphatase, it may be elevated in the serum of patients with bile duct diseases. Elevations in serum GGT, especially along with elevations in alkaline phosphatase, suggest bile duct disease. Measurement of GGT is an extremely sensitive test, however, and it may be elevated in virtually any liver disease and even sometimes in normal individuals. GGT is also induced by many drugs, including alcohol, therefore often when the ALP is normal a raised GGT can often (but not always) indicate alcohol use. Raised GGT can often be seen in cases of fatty liver and also where the patient consumes large amounts of Aspartame (artificial Sweetener) in diet drinks for example.

Bilirubin is the major breakdown product that results from the destruction of old red blood cells (as well as some other sources). It is removed from the blood by the liver, chemically modified by a process call conjugation, secreted into the bile, passed into the intestine and to some extent reabsorbed from the intestine. It is basically the pigment that gives faeces its brown colour. Bilirubin concentrations are elevated in the blood either by increased production, decreased uptake by the liver, decreased conjugation, decreased secretion from the liver or blockage of the bile ducts. In cases of increased production, decreased liver uptake or decreased conjugation, the unconjugated or so-called indirect bilirubin will be primarily elevated. In cases of decreased secretion from the liver or bile duct obstruction, the conjugated or so-called direct bilirubin will be primarily elevated.

Many different liver diseases, as well as conditions other than liver diseases (e.g. increased production by enhanced red blood cell destruction), can cause the serum bilirubin concentration to be elevated. Most adult acquired liver diseases cause impairment in bilirubin secretion from liver cells that cause the direct bilirubin to be elevated in the blood. In chronic, acquired liver diseases, the serum bilirubin concentration is usually normal until a significant amount of liver damage has occurred and cirrhosis is present. In acute liver disease, the bilirubin is usually increased relative to the severity of the acute process. In bile duct obstruction, or diseases of the bile ducts such as primary biliary cirrhosis or sclerosing cholangitis, the alkaline phosphatase and GGT activities are often elevated along with the direct bilirubin concentration.

Albumin is the major protein that circulates in the bloodstream. As it is made by the liver and secreted into the blood it is a sensitive marker and a valuable guide to the severity of liver disease. Low serum albumin concentrations indicate the liver is not synthesizing the protein and is therefore not functioning properly. The serum albumin concentration is usually normal in chronic liver diseases until cirrhosis and significant liver damage is present. There are many other proteins synthesized by the liver however the Albumin is easily, reliably and inexpensively measured.

Platelets are cells that form the primary mechanism in blood clots. They're also the smallest of blood cells. They derived from the bone marrow from the larger cells known as megakaryocytes. Individuals with liver disease develop a large spleen. As this process occurs platelets are trapped with in the sinusoids (small pathways within the spleen) of the spleen. While the trapping of platelets is a normal function for the spleen, in liver disease it becomes exaggerated because of the enlarged spleen (splenomegaly). Subsequently, the platelet count may become diminished.

The prothrombin time may be tested to evaluate disorders of blood clotting, usually bleeding. It is a broad screening test for many types of bleeding disorders. When the liver is damaged it may fail to produce blood clotting factors.

In a preferred embodiment, the endothelial generating cell, CD133+ cells and/or the hMSC preparation or composition is formulated in a matrix. The matrix may functionally mimic the extracellular matrix (ECM). The matrix may comprise PEG, fibrin, collagen, or a combination thereof. In an especially preferred embodiment, fibrinogen solutions that contain about 50 mg/mL to about 400 mg/mL fibrinogen and thrombin solutions containing about 250 units/mL to about 2000 units/mL thrombin are suitable for making a matrix. Typically, a fibrinogen solution containing about 120 mg/mL fibrinogen and a thrombin solution containing about 500 units/mL thrombin are used to make a fibrin-based matrix compatible with cell survival and function.

In certain embodiments, the matrix is sufficiently porous to allow cells to migrate out from it. In certain embodiments, the matrix allows growth factors to effuse. In certain embodiments, the matrix prevents cells from migrating out. In certain embodiments, these properties are controlled by the amount of PEG, fibrin, collagen, or a combination thereof present in the matrix. For example, increasing the amount of PEG, fibrin or collagen would decrease the permeability of the matrix.

In certain embodiments, PEGylated fibrin can be used for cell delivery and may additionally deliver therapeutic proteins or peptides of the application. A chemically active derivative of polyethylene glycol (benzotriazole carbonate (BTC)-PEG-BTC) can be utilized to PEGylate fibrinogen forming a porous gel that supports cell proliferation and differentiation. PEGylated fibrin system can bind any amine-containing protein or peptide in therapeutic applications. A PEGylated fibrin patch has been used for mesenchymal stem cell (MSC) transplantation by modifying fibrinogen (Fgn) with the benzotriazole carbonate derivative of PEG to create secondary crosslinking (WO03/093433; Zhang et al, Tissue Eng. 2006 January; 12(1):9-19; Liu et al., Am J Physiol Heart Circ Physiol. 2004 287: H501-H5011; herein incorporated by reference). The matrices of the application may be implanted in the subject such that they are in physical contact with the liver.

In one embodiment, the EGCs, such as the AC133+ cells, may be administered to the subject via a matrix that comprises hepatocytes or hepatocytes progenitor cells. Matrices comprising hepatocytes or hepatocytes progenitor cells are known in the art (see for example, Takimoto et al. (2003) De novo liver tissue formation in rats using a novel collagen-polypropylene scaffold; Cell Transplant.; 12(4):413-21; Elcin et al. (1997) Xenotransplantation of fetal porcine hepatocytes in rats using a tissue engineering approach, Artif Organs. 23(2):146-52; Elcin et al., (1998) Artif Organs.; 22(10):837-46; Risbud et al. (2003) Hydrogel-coated textile scaffolds as candidate in liver tissue engineering: II. Evaluation of spheroid formation and viability of hepatocytes. J Biomater Sci Polym Ed.; 14(7):719-31; Lin et al. (2004) Assessing porcine liver-derived biomatrix for hepatic tissue engineering. Tissue Eng.; 10(7-8):1046-53.)

In one embodiment, the endothelial generating cells such as the CD133+ cells are co-administered simultaneously with the a second cell population, such as hMSCs. In another embodiment the second cell population are administered before or after the injection of the endothelial generating cells. Administration of the EGCs or the second cell population may be carried out using the same mode or a different mode of administration. For example, AC133+ cells can be administered via the Edmonton protocol, while hepatocytes or MSCs might be administered intravenously.

In one embodiment of the methods described herein, a recombinant polypeptide or a drug is administered to the subject in combination with the administration of cells. The polypeptide or drug may be administered to the subject before, concurrently, or after the administration of the cells. In one preferred embodiment, the recombinant polypeptide or drug promotes angiogenesis, vasculogenesis, tissue regeneration or both. In another embodiment, the recombinant polypeptide or drug promotes the proliferation or differentiation of the endothelial generating cells, of the mesenchymal stem cells, of hepatocytes, or of both. In one embodiment, the recombinant polypeptide is VEGF, BFGF, SDF, CXCR-4 or CXCR-5, or a fragment thereof which retains a therapeutic activity to the ischemic tissue. In one embodiments, the recombinant polypeptide is hepatocyte growth factor, transforming growth factor, epidermal growth factor, tumor necrosis factor-alpha, or interleukins-1 and -6, or a fragment thereof which retains a regenerative activity in the ischemic tissue.

In one specific embodiment of the present invention, soluble polypeptides are linked to nonpolypeptide polymers, such as the matrix of previously described embodiments. In one specific embodiment, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ₋₁CH₂CH₂OH (1), where n is 20 to 2300 and X is H or a terminal modification, e.g., a C₁₋₄ alkyl. In one embodiment, the PEG of the invention terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-0473084 and U.S. Pat. No. 5,932,462. One form of PEGs includes two PEG side-chains (PEG2) linked via the primary amino groups of a lysine (Monfardini, C., et al., Bioconjugate Chem. 6 (1995) 62-69).

PEG conjugation to peptides or polypeptides generally involves the activation of PEG and coupling of the activated PEG-intermediates directly to target polypeptides/peptides or to a linker, which is subsequently activated and coupled to target polypeptides/peptides (see Abuchowski, A. et al, J. Biol. Chem., 252, 3571 (1977) and J. Biol. Chem., 252, 3582 (1977), Zalipsky, et al., and Harris et. al., in: Poly(ethylene glycol) Chemistry: Biotechnical and Biomedical Applications; (J. M. Harris ed.) Plenum Press: New York, 1992; Chap. 21 and 22).

One skilled in the art can select a suitable molecular mass for PEG, e.g., based on how the pegylated polypeptide is used therapeutically, the desired dosage, circulation time, resistance to proteolysis, immunogenicity, and other considerations. For a discussion of PEG and its use to enhance the properties of polypeptides, see N. V. Katre, Advanced Drug Delivery Reviews 10: 91-114 (1993).

In one embodiment of the invention, PEG molecules may be activated to react with amino groups on the soluble polypeptides, such as with lysines (Bencham C. O. et al., Anal. Biochem., 131, 25 (1983); Veronese, F. M. et al., Appl. Biochem., 11, 141 (1985); Zalipsky, S. et al., Polymeric Drugs and Drug Delivery Systems, adrs 9-110 ACS Symposium Series 469 (1999); Zalipsky, S. et al., Europ. Polym. J., 19, 1177-1183 (1983); Delgado, C. et al., Biotechnology and Applied Biochemistry, 12, 119-128 (1990)). In another embodiment, PEG molecules may be coupled to sulfhydryl groups on the polypeptides (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. Nos. 6,610,281 and 5,766,897 describes exemplary reactive PEG species that may be coupled to sulfhydryl groups. In some embodiments, the pegylated polypeptides comprise a PEG molecule covalently attached to the alpha amino group of the N-terminal amino acid. Site specific N-terminal reductive amination is described in Pepinsky et al., (2001) JPET, 297,1059, and U.S. Pat. No. 5,824,784. The use of a PEG-aldehyde for the reductive amination of a polypeptide utilizing other available nucleophilic amino groups is described in U.S. Pat. No. 4,002,531, in Wieder et al., (1979) J. Biol. Chem. 254,12579, and in Chamow et al., (1994) Bioconjugate Chem. 5, 133.

In particular, the invention methods are useful for therapeutic vasculogenesis for the treatment of a liver in a subject in need thereof. Administration of CD133+ cells and hMSCs according to invention methods can be used as a sole treatment or as an adjunct to surgical and/or medical treatment modalities. For example, the methods described herein for treatment of liver disease can be used in conjunction with a diaretic agent, ursodeoxycholic acid, an anti-nausea agent, or a combination thereof. Additional liver disease treatments can be found in Harrison's Principles of Internal Medicine 15th Edition, Braunwald et al, 2001, McGraw-Hill, U.S.A., and THE MERCK MANUAL, 17TH Edition, (Beers and Berkow, Ed.), 1999, Whitehouse Station, N.J. incorporated herein by reference.

The therapeutically effective amount of the CD133+ cells is a maximum number of cells that is safely received by the subject. The minimum number of cells necessary for induction of new blood vessel formation in the liver can be determined empirically, without undue experimentation, by dose escalation studies. For example, such a dose escalation could begin with approximately 10⁴/kg body weight of CD133+ cells alone, or in combination with approximately 10⁴/kg hMSCs. Effective amounts of CD133+ cells sufficient to cause the desired neovascularization can be estimated based on animal data using routine computational methods. In one embodiment the effective amount is about 1.5×10⁵ CD133+ cells per kg body mass to about 3×10⁵ per kg body mass. In another embodiment the effective amount is about 3×10⁵ per kg body mass to about 4.5×10⁵ CD133+ cells per kg body mass. In another embodiment the effective amount is about 4.5×10⁵ per kg body mass to about 5.5×10⁵ CD133+ cells per kg body mass. In another embodiment the effective amount is about 5.5×10⁵ per kg body mass to about 7×10⁵ CD133+ cells per kg body mass. In another embodiment the effective amount is about 7×10⁵ per kg body mass to about 1×10⁶ CD133+ cells per kg body mass. In another embodiment the effective amount is about 1×10⁶ per kg body mass to about 1.5×10⁶ CD133+ cells per kg body mass. In one embodiment the effective amount of human CD133+ cells is between about 1.5×10⁶ and 4.5×10⁶ CD133+ cells per kg of the subject's body mass and In a preferred embodiment the effective amount is about 5×10⁵ CD133+ cells per kg of the subject's body mass.

In some embodiments of the methods described herein, the composition comprising the CD133+ cells is introduced into a vessel of the subject without substantially altering the arterial pressure. In other embodiments, the composition is introduced into a vessel by blocking arterial flow for an amount of time, such as from 5 seconds to two minutes, such that the injected cells can pool and adhere to the vessel. In one embodiment, a balloon catheter is used to allow pressure driven administration.

One aspect of the invention further provides a pharmaceutical formulation, comprising: (a) CD133⁺/CD34⁺ cells enriched from umbilical cord blood; (b) mesenchymal stem cells containing surface antigens identified by monoclonal antibodies SH2, SH3 or SH4 enriched from bone marrow; and (c) a pharmaceutically acceptable carrier. In some embodiments, the formulation comprises from 10⁴ to 10⁹ CD133⁺/CD34⁺ cells. In another embodiment, the composition comprises from 10⁴ to 10⁹ mesenchymal stem cells. In a further embodiment, the formulation is prepared for administration by a catheter.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

### EXEMPLIFICATION

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other stem cell sources and selection methods, other culture media and culture methods, other dosage and treatment schedules, and other animals and/or humans, all without limitation, can be employed, without departing from the scope of the invention as claimed.

### Example 1

### In Vitro Studies of the Effects of CD133+ Cells on Co-Cultured Hepatocytes

Mononuclear cells are isolated by Ficoll density gradient centrifugation as described earlier. Cells are then be incubated with either CD133+ or CD31+ (terminally differentiated endothelial cell as a control) specific magnetic beads per manufactures instructions (AutoMACS, Miltenyi). CD133+ selected from UCB or bone marrow is enumerated and characterized by flow cytometry. Selected cells are characterized by specific surface markers, including CD133, CD34, CD31, CXCR2/CXCR1 (IL-8 receptors), and KDR (VEGFR2) to determine purity and surface characteristics of cells after bead selection.

Human hepatocytes are purchased from Cambrex, (Cambrex, Md.) and propagated in DMEM media. NOD/SCID mice hepatocytes are isolated by a modified two-step collagenase perfusion of the liver as described (see Fiegel et al., Tissue Eng. 6, 619-26 (2000)). 10 rats are anesthetized using a mixture of Hypnorm (0.3 mg/mouse given intraperitoneally) and Dormicum (12.5 mg/mouse given intraperitoneally). After cannulation of the portal vein the liver is perfused with 200 mL Calcium and Magnesium free Hanks balanced saline solution (37° C.; 10 mL/min). After 10 minutes the liver is perfused with the same buffer supplemented with 0.05% collagenase (Boehringer Mannheim) and 2.5 mmol/L CaCl₂ for 10 minutes. After the entire liver is resected, the lobes are thoroughly cut in small pieces and diluted in medium (HBBS supplemented with 2.5% BSA) and 2.5 mmol/L CaCl₂. Dispersed cells are centrifuged (50G˜600 rpm) three times for 3 minutes. Cell viability is checked by trypan blue exclusion. Hepatocytes are plated at a density of 8×10³/cm² in Williams medium supplemented with 10% heat-inactivated newborn calf serum (Gibco Life Technologies Inc., Rockville, Md.) and incubated at 37° C. for 4 hours before co-culture experiments.

NOD/SCID mice hepatocytes and human CD133+ cells are cultured in hepatocyte culture medium (Gibco/BRL, Rockville, Md.) containing 10% fetal calf serum in 6-well transwell plates (Nunc, USA). Twenty-four hours after plating the medium is removed and replaced by hepatocyte culture medium containing 15 mmol/L CCl₄ for 45 minutes. NOD/SCID mice are injected intraperitoneally with a single dose of 0.4 ml of CCl₄/kg body weight CCl₄ diluted to 100 μl with olive oil to induce acute liver damage NOD/SCID mice injured hepatocytes are isolated as described and used in co-culture experiments. The cell survival rate is estimated by the trypan blue exclusion test.

Following 24, 48 and 72 hours of co-culture each well, cells is washed in phosphate-buffered saline. The cells are then incubated in serum-free DMEM to which MTT (0.5 mg ml⁻¹) is added to check the proliferative capacity of injured hepatocytes, and incubated for a further 4 h. Then the medium is removed and the cells are incubated for 15 min with 100 μl of acidic isopropanol (0.08 N HCl) to dissolve the formazan crystals. The absorbance of the MTT formazan is determined at 570 nm in an enzyme-linked immunosorbent assay (ELISA) reader. Viability is defined as the ratio (expressed as a percentage) of absorbance of treated cells to untreated cells and co-cultured cells to single cell culture.

The albumin levels in co-culture is measured using ELISA. In brief, supernatants are incubated in 96-well microtiter plates for 1 h at 37° C. or overnight at 4° C. After washing in PBS, non-specific binding sites are blocked in PBS containing 0.5% bovine serum albumin (BSA) for 1 h at room temperature. After another washing step in PBS, peroxidase-conjugated goat anti-mouse albumin antibody is added in PBS containing 1% BSA and incubated for 2 h at room temperature. Following washing, the substrate (0.5 mg 2,2-azino-di-3-ethylbenzothiazoline-6-sulfonic acid per ml of 100 mM sodium acetate, 50 mM sodium phosphate and 9×10⁻³% H₂O₂) is added and the absorption is measured at 405 nm in an ELISA reader. All ELISA determinations are carried out in duplicates. The concentrations of Alfa-fetoprotein (AFP), urea and CytP450 are examined by ELISA assay kits. Apoptotic activity is analyzed by direct labeling followed by Flow or western blotting using anti-BCL2, anti BAK and anti-BAX (Upstate, NY). Level of VEGF, PDGF and HGF is also tested using ELISA ((R&D systems, Minneapolis, Minn.) kits.

Immunostaining is employed to detect mouse albumin and cytokeratin (CK) 8 to assess the change in functionality of co-cultured hepatocytes. For mouse albumin, cells in culture for 35 days are fixed with 3% paraformaldehyde/2% sucrose/PBS at room temperature for 10 minutes. Fixed cells are permeabilized with 0.5% Triton X-100 (Sigma) in PBS for 5 minutes and then incubated in 3% BSA/PBS for 10 minutes. Dishes are sequentially incubated with rabbit polyclonal anti-mouse albumin antibodies (Sigma, St Louis) in 1% BSA/PBS at a 1:500 dilution for 10 minutes at room temperature followed by secondary antibodies: Cy3-conjugated goat anti-rabbit IgG (The Jackson ImmunoResearch Laboratories Inc., West Grove, Pa., USA) in 1% BSA/PBS at a 1:500 dilution. Finally, the Cy3 signal is detected at 520 nm and 550 nm using a U-MWIG filter (Olympus Optical Co., Tokyo, Japan). Photomicrographs are taken using a CCD camera DP 50 (Olympus Optical Co.).

Reverse transcription polymerase chain reaction (RT-PCR) analyses is performed using purified RNA co-cultured Hepatocytes harvested on days 0, 7, 14, and 21. Total RNA is extracted using Trizol reagent (Invitrogen, Carlsbad, Calif.), and reverse-transcribed with random primer using Superscript II Reverse Transcriptase (Invitrogen, Carlsbad, Calif.) according to manufacturer's instructions. Resultant cDNA products are amplified for Thy-1, AFP, albumin, CK 19, Mac-1, and TER119 genes using commercially available primers and the conditions: at 94° C. for 5 minutes followed by 35 cycles at 94° C. for 30 seconds, 60° C. for 30 seconds, and then a final cycle at 72° C. for 10 minutes.

After co-culture of 7 hours, cells are examined for differentiation status of CD133 using antibodies against CD31, CD45, (R&D systems, Minneapolis, Minn.) by immunohistochemical staining.

### Example 2

### Treatment of a Mouse Model of Liver Injury with CD133+ Cells

Eight-week old male/female non-obese diabetic/severe combined immunodeficiency (NOD/SCID) mice from the Jackson laboratories are used in all experiments. Mice are housed at a controlled temperature (20° C.) under 12 h/12 h light-dark conditions and are maintained on standard diet with freely available water. All procedures are performed in accordance with Institutional Animal Care and Use Committee approval. Animals are divided into 6 groups with 10 mice each, calculated based on ANOVA calculation.
Group 1: NOD/SCID mice with allyl alcohol (AA)-induced liver injury, injected with PBS [n=10];
Group 2: NOD/SCID mice with carbon tetrachloride (CCl₄)-induced liver injury, injected with PBS [n=10];
Group 3: AA-induced liver injury NOD/SCID mice transplanted with CD133+ cells from human bone marrow [n=10];
Group 4: AA-induced liver injury NOD/SCID mice transplanted with CD133+ cells from human umbilical cord blood [n=10]
Group 5: CCl₄-induced liver injury NOD/SCID mice transplanted with CD133+ cells from human bone marrow [n=10];
Group 6: CCl₄-induced liver injury NOD/SCID mice transplanted with CD133+ cells from human umbilical cord blood [n=10];

In AA-induced liver injury model, allyl alcohol is injected intraperitoneally 24 h before cell transplantation. As for CCl₄-induced liver injury model, 1 ml/kg carbon tetrachloride (CCl₄) dissolved in olive oil (1:1) is administrated intraperitoneally into mice twice a week for 4 weeks. One day (24 hours) after the eighth injection of CCl₄, 1×10⁵ CD133+ cells either from bone marrow or cord blood are transplanted via portal vein. Mice are sacrificed at 0, 7, 14, 21, 28, and 56 days after CD133+ transplantation. A separated experiment is carried out to examine the survival rate.

Human cord blood-derived CD133 cells (0.1-1×10⁶) is infused by lateral tail vein injection to the different groups as described earlier. At 3 weeks after transplantation, mice are screened for cell engraftment by detection of PKH26-positive cells or human leukocyte antigen-expressing cells in peripheral blood using fluorescence-activated cell sorting analysis. Eight weeks after induction of liver injury and cell transplantation the recipient NOD-SCID mice are sacrificed. Peripheral blood and BM from all mice are analyzed for donor-derived positive blood cells or for human cell engraftment as described below. Before removal of liver, pancreas, heart, intestine, and spleen each mouse is perfused with saline by cardiac puncture under anesthesia to wash out circulating blood cells in the organs.

CD133+ cells are labeled with PKH26 (Sigma, St Louis) and the cells are followed for differentiation, proliferation and retention as described earlier. The liver tissue is evaluated for the presence of donor-derived cells (CD133+) with hepatocytes, smooth muscle (SM), and endothelial phenotype using combination of immunohistochemistry and western blot analysis. The increased vessel density within the injured liver is analyzed by H&E staining. Immunofluorecence microscopy: Imaging is performed using confocal microscopy. Tissues are double labeled with antibodies against liver specific proteins such as albumin, Alfa-fetoprotein (AFP) and total protein. Some common enzymes used to detect liver disease are alanine aminotransferase (ALT), alkaline phosphatase (ALP), aspartate aminotransferase (AST) and gamma-glutamyl transferase (GGT) are analyzed to monitor changes in liver function.

Hepatocyte apoptosis and proliferation are determined by tunnel staining using a commercial kit. Expression of apoptosis related genes inducing Bcl-2, Bcl-x and Bax are examined by western blot and quantitative RT-PCR after stem cell transplantation. Proliferation is assessed by staining with antibodies against Ki67, with or without BrdU labeling.

For FISH analysis, paraffin-embedded liver samples are incubated with paraffin pretreatment solution (Vysis, Downers Grove, Ill., http://www.vysis.com) at 85° C. for 30 minutes and with protease at 37° C. for 10 minutes. After DNA denaturation in 70% formamide at 75° C. for 5 minutes, the specimens are incubated with species-specific probes for 14-18 hours at 37° C. A Spectrum Orange- or Spectrum Green-conjugated human X-chromosome probe (Vysis) or a Cy-3-conjugated human centromere probe (Cambio Ltd., Cambridge, U.K., http://www.cambio.co.uk) is used for detecting human cells and a FITC-conjugated murine centromere probe (Cambio Ltd.) is used for detecting mouse cells. To count total numbers of human cells and to exclude any autofluorescence, nuclei are stained with 4′,6-diamidino-2-phenylindole (DAPI) (Vector Laboratories, Burlingame, Calif., http://www.vectorlabs.com).

Transplanted stem cells are examined for differentiation into vessels containing endothelial cells. First the engrafted area is examined using high power magnification with H&E staining to evaluate formed vessels. The appearance of PKH26 in the cells forming the new vessels indicates that the vessels are formed from the grafted stem cells. The differentiation of stem cells into endothelial cells within the vessels is monitored by immunohistochemistry or immunofluorescence using antibodies against Von Willebrand factor, Flt1, Tie, Tek, PECAM, and P and E-selectins. Antibodies against smooth muscle myosin heavy chain, smooth muscle β-actin, and desmin are used to monitor differentiation to smooth muscle. Blood flow in the liver is also monitored using laser Doppler.

For CD133-cell induced intrahepatic vascularization studies, immunohistochemical staining are performed on acetone-fixed 5-μm cryosections of hepatic tissue. The sections are incubated in PBS containing 1.5% hydrogen peroxide at room temperature for 15 minutes to block endogenous peroxidase activity. The sections are stained with anti-CD31 mAbs (Dako, USA) for 60 minutes, followed by fixation in phosphate buffered formaldehyde 3.7% (10 minutes). 30 Sections are incubated for 1 hour with a horseradish peroxidase-coupled rabbit anti-mouse antibody. After three washes, labeling is visualized with 0.03% 3,3′-diaminobenzidine tetrahydrochloride (DAB) solution containing 0.1% hydrogen peroxide for 10 minutes. Sections are counterstained for 45 seconds in Mayer's hematoxylin and mounted in a synthetic resin (DPX). The microvascular density (MVD) is determined by analyzing at least four independent microscopic fields per tissue section and at least two sections per liver remnant. Fields containing large microvascular structures (portal/central venules and hepatic arterioles) are excluded, and only microvessels are counted. The number of microvessels stained for CD31 is counted by two independent observers. MVD is expressed as the mean number of microvessels.

For detection of liver-specific antigens albumin, Hep Par 1, CK18, and CK8 a standard immunohistochemical technique using formalin-fixed and paraffin-embedded tissue are used. The primary antibodies are diluted in PBS, pH 7.4, and applied at a dilution of 1:100 for the anti-mouse hepatocyte antibody (Hep Par 1, DAKO) at 1:50 for the anti-mouse albumin (DAKO), anti-mouse antibody (DAKO), and anti-mouse CK8 (DAKO). After application of the secondary antibody (DAKO) color development is performed with the 3-amino-9-ethylcarbazole chromogen system (Zymed, USA) for Hep Par 1 and with diaminobenzidine (DAKO) for albumin, CK18, and CK8.

To further confirm the immunohistochemistry findings, RT-PCR analysis is performed using total RNA from liver tissue isolated using Trizol reagent (GIBCO/BRL, MD). Mouse-specific primers are used to detect expression of albumin, AFP, cytochrome P-450, CK8, CK18, and CK19 mRNA in the mouse liver tissues following first strand synthesis using cDNA synthesis kit (Strategene, SanDiago, Calif.). Expression of 13-tubulin mRNA is used as a housekeeping gene. The PCR conditions are 30 seconds at 94° C., 45 seconds at 55° C., 45 seconds at 72° C., and for all the genes appropriate conditions are standardized with proper control.

It is expected that the mice with AA-induced or CCl₄-induced liver injury will show an enhancement in liver function and liver vascularization in response to these therapeutic treatments.

### Example 3

### Treatment of Liver Disease with CD133+ Cells

A patient with liver disease such as alcohol induced, chemically induced, virally induced, and genetically influenced liver disease is eligible for treatment. The patient is screened within 30 days of scheduling of treatment. Hepatic angiography is evaluated for anatomy favorable for the treatment protocol.

Once hepatic anatomy in the eligible patient is determined, the patient undergoes bone marrow aspiration as described in U.S. Patent Publication Nos. 2004/0258670, 2005/0069527. CD133⁺ cells are isolated from the MNCs according to the method described in U.S. Patent Publication Nos. 2004/0258670 and 2005/0069527, by labeling with CD133⁺-conjugated magnetic beads followed by automated sorting through magnetic columns (Automacs, Miltenyi). The selected CD133⁺ cells are then washed in buffer solution and can be stored in a concentrated solution of 5 ml. normal saline.

After the patient is given time to recover from the bone marrow aspiration, a complete blood count (CBC), chest X-ray, liver function tests (LFTs), and coagulation screen are performed. The transplants are carried out under local anaesthetic. A very thin needle is used, along with x-rays, to locate the main blood vessel in the liver (the portal vein) Next portal vein cannulation is performed. When the portal vein is cannulated, approximately 1×10⁴ to 1×10⁵ of the isolated CD133⁺ cells in 250 ml of medium in an intravenous fluid bag are allowed to infuse under gravity pressure. Portal pressure is monitored during and after infusion of 5 ml of the CD133+ cells, after each subsequent milliliter of cells, and again when the transplant is completed. To minimize the risk of bleeding, the catheter tract is plugged with coils and Tisseel. Other therapeutic cells such as mesenchymal stem cells, hepatocytes or hepatocytes progenitor cells, and/or growth factors such as PDGF are also administered to a subset of patients in increasing dosages, either alone or in combination with the CD133+ cells.

Patients are discharged the following day when an ultrasound has confirmed the absence of any portal vein thrombosis or intraperitoneal bleed and that the CBC and LFTs are acceptable. Aspirin (81 mg/day for 14 days) and enoxaparin (30 mg b.i.d. s.c. for 10 days) are prescribed once major bleeding had been excluded. Liver function is monitored closely after transplant.

Patients are seen in follow up every month initially and longer term every 3 to 6 months as required. Liver function, immunosuppressive levels and adverse events are monitored regularly. Patients are monitored for complications of liver function as per standard guidelines. LFTs are performed every 3 months initially and then every 6 months once stable. It is expected that subjects with liver disease such as alcohol induced, chemically induced, virally induced, and genetically influenced liver disease will show improvement in one or more markers of liver function in response to the therapies described in this example.

We claim:

1. A method for improving liver function in a subject in need thereof, comprising administering to said subject a composition comprising AC133+ cells.

2. A method of treating liver disease in a subject in need thereof, comprising administering to said subject a composition comprising AC133+ cells.

3. The method of claim 2, wherein administering of the composition to the subject reduces one or more symptoms of the liver disease.

4. The method of claim 3, wherein the reduced symptoms are selected from loss of liver function, ischemia, fibrosis and cirrhosis.

5. The method of claim 2, wherein the subject is afflicted with liver ischemia, liver fibrosis, liver cirrhosis, acute liver failure, Alagille syndrome, alcohol liver disease, Alpha 1—antitrypsin deficiency, autoimmune hepatitis, biliary atresia, chronic hepatitis, cirrhosis, cholestatic liver disease, cystic disease of the liver, fatty liver, galactosemia, gallstones, Gilbert's syndrome, hemochromatosis, hepatitis A, hepatitis B, hepatitis C, liver cancer, neonatal hepatitis, non-alcoholic liver disease, non-alcoholic steatohepatitis, porphyria, primary biliary cirrhosis, primary sclerosing cholangitis, Reye's syndrome, sarcoidosis, steatohepatitis, tyrosinemia, type I glycogen storage disease, viral hepatitis, Wilson's disease, or any combination thereof.

6. The method of claim 5, wherein the subject is afflicted with liver cirrhosis.

7. The method of claim 6, wherein the liver cirrhosis is selected from micronodular, macronodular and mixed cirrhosis.

8. The method of claim 2, wherein the subject is a liver transplant donor or a liver transplant recipient.

9. The method of claim 2, wherein the subject is a mammal.

10. The method of claim 2, wherein the subject is human.

11. The method of claim 2, wherein the AC133+ cells are isolated from umbilical cord blood, bone marrow or peripheral blood.

12. The method of claim 2, wherein the AC133+ cells are autologous to the subject.

13. The method of claim 2, wherein the AC133+ cells are allogeneic to the subject.

14. The method of claim 2, wherein the AC133+ cells are administered by infusion into an artery, via the Edmonton protocol, or via direct application.

15. The method of claim 2, wherein the composition comprises a matrix in which the cells are embedded.

16. The method of claim 15, wherein the matrix comprises: polyethylene glycol (PEG), collagen, fibrin, or a combination thereof.

17. The method of claim 2, wherein the composition further comprises soluble human fibronectin, hyaluronan or type I collagen, or a combination thereof.

18. The method of claim 2, wherein the composition further comprises mesenchymal stem cells, hepatocytes or hepatocytes progenitor cells.

19. The method of claim 2, wherein the composition further comprises administering to the subject a cytokine, chemokine or growth factor.

20. The method of claim 19, wherein the growth factor is bFGF or VEGF.

21. The method of claim 2, wherein the AC133+ cells are selected from AC133+CD34-cells, AC133+CD34+ cells, or combinations thereof.

22. The method of claim 21, wherein the AC133+ cells are AC133+CD34+KDR-CXCR4− cells.

23. The method of claim 21, wherein at least 10% of cells in the composition are AC133+ cells.

24. The method of claim 2, wherein the AC133+ cells are expanded in vitro prior to administering to the subject.

25. The method of claim 2, wherein administering of the composition reduces, delays or eliminates the need for liver transplantation.

26. The method of claim 2, wherein administering of the composition increases the likelihood of survival for one year by at least 25% to allow for liver transplantation.

27. An implantable matrix comprising a plurality of AC133+ cells.

28. The matrix of claim 27, wherein the matrix comprises: polyethylene glycol (PEG), collagen, fibrin, or any combination thereof.

29. The matrix of claim 28, wherein the fibrin matrix is polymerized from a solution that contains 50 mg/ml to 400 mg/ml fibrinogen and 250 units/mL to 2000 units/mL thrombin.

30. The matrix of claim 27, further comprising mesenchymal stem cells, hepatocytes, hepatocytes progenitor cells, or any combination thereof.

31. The matrix of claim 27, further comprising a recombinant peptide.

32. The matrix of claim 31, wherein the recombinant peptide is selected from: transforming growth factor, tumor necrosis factor-alpha, basic fibroblast growth factor (bFGF), CX chemokine receptor (CXCR)-4, CXCR-5, vascular endothelial growth factor (VEGF), hepatocyte growth factor (HGF), fibroblast growth factor (FGF), stromal cell-derived factor 1 (SDF-1), angiopoietin-, leukemia inhibitory factor, interleukins IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), FMS-like tyrosine kinase 3 (Flt3)-ligand, B cell activating factor, artemin, bone morphogenic protein factors, epidermal growth factor (EGF), glial derived neurotrophic factor, lymphotactin, macrophage inflammatory proteins, myostatin, neurturin, nerve growth factors, platelet derived growth factors, placental growth factor, pleiotrophin, stem cell factor, or any combination thereof.

33. The method of claim 1, wherein improving liver function comprises increasing serum levels of albumin, platelet counts, or both.

34. The method of claim 1, wherein improving liver function comprises decreasing serum levels of alanine aminotransferase (ALT), aspartate aminotransferase (AST), gamma glutamic transpeptidase (GGT), alkaline phosphatase (ALP), bilirubin, or decreasing the results of a prothrombin time test, or any combination thereof.
